(19) United States
(12) Patent Application Publication
(10) Pub. No.: US 2020/0143534 A1
(43) Pub. Date: May 7, 2020

(54) MEDICAL IMAGING SYSTEM, METHOD AND COMPUTER PROGRAM PRODUCT

(71) Applicant: Sony Corporation, Tokyo (JP)

(72) Inventors: Christopher WRIGHT, London (GB); Matthew LAWRENSON, Bussigny (CH); Nicholas WALKER, Middlesex (GB); Akinori KAMODA, Tokyo (JP)

(73) Assignee: Sony Corporation, Tokyo (JP)

(21) Appl. No.: 16/619,481

(22) PCT Filed: May 28, 2018
(86) PCT No.: PCT/JP2018/020317

§ 371 Date: Dec. 5, 2019

(30) Foreign Application Priority Data
Jun. 29, 2017 (EP) 17178724.5

(51) Int. Cl. G06T 7/00; A61B 5/00; A61B 1/00

(52) U.S. Cl. CPC G06T 7/0012; A61B 5/0051; G06T 2207/30101; A61B 1/00009; A61B 5/0095

(57) ABSTRACT

A medical imaging system including circuitry configured to: apply a surface acoustic wave on the tissue to interact with the vessel; capture an image of the tissue when the surface acoustic wave interacts with the vessel; and identify a property of the vessel from the captured image.

### CROSS REFERENCE TO RELATED APPLICATIONS

This application claims the benefit of EP17178724.5 filed Jun. 29, 2017, the entire contents of which are incorporated herein by reference.

### TECHNICAL FIELD

The present disclosure relates to a medical imaging system, method and computer program.

### BACKGROUND ART

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present disclosure.

It is sometimes necessary to produce an image of vasculature at image depths of several millimetres during surgical procedures (such as procedures including endoscopy and microscopy). This can be done using Laser Speckling techniques.

However, this technique lacks depth resolution. This can cause errors when estimating the diameter of vessels and the flow within them.

Furthermore, as Laser Speckle Contrast Imaging (LSCI) is also used during and prior to delicate surgical procedures, the accuracy of planned interactions with the tissue, such as a surgical incision, may also be limited.

It is an aim of the present disclosure to address at least these issues.

### SUMMARY

According to embodiments of the disclosure, there is provided a medical imaging system including circuitry configured to: apply a surface acoustic wave on the tissue to interact with the vessel; capture an image of the tissue when the surface acoustic wave interacts with the vessel; and identify a property of the vessel from the captured image.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 is a view depicting an example of a schematic configuration of an endoscopic surgery system.

FIG. 2 is a block diagram depicting an example of a functional configuration of a camera head and a camera control unit (CCU) depicted in FIG. 1.

FIG. 3 an embodiment of the disclosure is shown.

FIG. 4A shows an endoscope view according to embodiments of the disclosure.

FIG. 4B shows an endoscope view according to embodiments of the disclosure.

FIG. 4C shows an endoscope view according to embodiments of the disclosure.

FIG. 4D shows an endoscope view according to embodiments of the disclosure.

FIG. 5 shows the endoscope view according to embodiments of the disclosure.

FIG. 6 shows a data structure according to embodiments of the disclosure.

FIG. 7 shows a look up table according to embodiments of the disclosure.

FIG. 8 shows an endoscope according to embodiments of the disclosure.

FIG. 9 shows the interaction of a SAW wave on a blood vessel according to embodiments of the disclosure.

FIG. 10 shows a flowchart according to embodiments of the disclosure.

FIG. 11 shows a flowchart according to embodiments of the disclosure.

FIG. 12 shows a flowchart according to embodiments of the disclosure.

### DESCRIPTION OF EMBODIMENTS

Referring now to the drawings, wherein like reference numerals designate identical or corresponding parts throughout the several views.

1. Application

<<1. Application>>

The technology according to an embodiment of the present disclosure can be applied to various products. For example, the technology according to an embodiment of the present disclosure may be applied to an endoscopic surgery system.

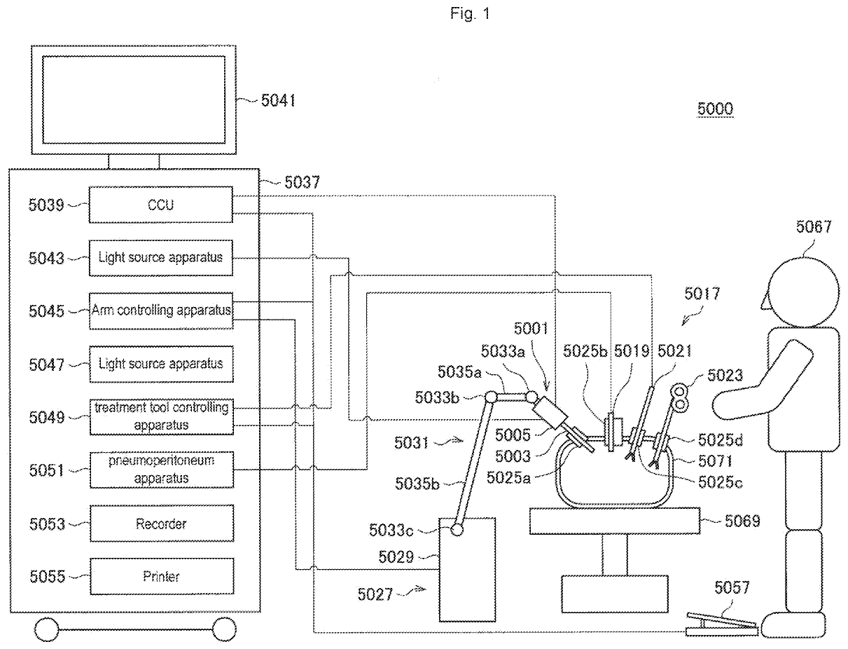

FIG. 1 is a view depicting an example of a schematic configuration of an endoscopic surgery system 5000 to which the technology according to an embodiment of the present disclosure can be applied. In FIG. 1, a state is illustrated in which a surgeon (medical doctor) 5067 is using the endoscopic surgery system 5000 to perform surgery for a patient 5071 on a patient bed 5069. As depicted, the endoscopic surgery system 5000 includes an endoscope 5001, other surgical tools 5017, a supporting arm apparatus 5027 which supports the endoscope 5001 thereon, and a cart 5037 on which various apparatus for endoscopic surgery are mounted.

In endoscopic surgery, in place of incision of the abdominal wall to perform laparotomy, a plurality of tubular aperture devices called trocars 5025a to 5025d are used to puncture the abdominal wall. Then, a lens barrel 5003 of the endoscope 5001 and the other surgical tools 5017 are inserted into body lumens of the patient 5071 through the trocars 5025a to 5025d. In the example depicted, as the other surgical tools 5017, a pneumoperitoneum tube 5019, an energy treatment tool 5021 and forceps 5023 are inserted into body lumens of the patient 5071. Further, the energy treatment tool 5021 is a treatment tool for performing incision and peeling of a tissue, sealing of a blood vessel or the like by high frequency current or ultrasonic vibration. However, the surgical tools 5017 depicted are mere examples at all, and as the surgical tools 5017, various surgical tools which are generally used in endoscopic surgery such as, for example, a pair of tweezers or a retractor may be used.

An image of a surgical region in a body lumen of the patient 5071 imaged by the endoscope 5001 is displayed on a display apparatus 5041. The surgeon 5067 would use the energy treatment tool 5021 or the forceps 5023 while watching the image of the surgical region displayed on the display apparatus 5041 on the real time basis to perform such treatment as, for example, resection of an affected area. It is to be noted that, though not depicted, the pneumoperitoneum tube 5019, the energy treatment tool 5021 and the forceps 5023 are supported by the surgeon 5067, an assistant or the like during surgery.

(Supporting Arm Apparatus)

The supporting arm apparatus 5027 includes an arm unit 5031 extending from a base unit 5029. In the example depicted, the arm unit 5031 includes joint portions 5033a, 5033b and 5033c and links 5035a and 5035b and is driven under the control of an arm controlling apparatus 5045. The endoscope 5001 is supported by the arm unit 5031 such that the position and the posture of the endoscope 5001 are controlled. Consequently, stable fixation in position of the endoscope 5001 can be implemented.

(Endoscope)

The endoscope 5001 includes the lens barrel 5003 which has a region of a predetermined length from a distal end thereof to be inserted into a body lumen of the patient 5071, and a camera head 5005 connected to a proximal end of the lens barrel 5003. In the example depicted, the endoscope 5001 is depicted which includes as a hard mirror having the lens barrel 5003 of the hard type. However, the endoscope 5001 may otherwise be configured as a soft mirror having the lens barrel 5003 of the soft type.

The lens barrel 5003 has, at a distal end thereof, an opening in which an objective lens is fitted. A light source apparatus 5043 is connected to the endoscope 5001 such that light generated by the light source apparatus 5043 is introduced to a distal end of the lens barrel by a light guide extending in the inside of the lens barrel 5003 and is irradiated toward an observation target in a body lumen of the patient 5071 through the objective lens. It is to be noted that the endoscope 5001 may be a direct view mirror or may be a perspective view mirror or a side view mirror.

An optical system and an image pickup element are provided in the inside of the camera head 5005 such that reflected light (observation light) from an observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 5039. It is to be noted that the camera head 5005 has a function incorporated therein for suitably driving the optical system of the camera head 5005 to adjust the magnification and the focal distance.

It is to be noted that, in order to establish compatibility with, for example, a stereoscopic vision (three dimensional (3D) display), a plurality of image pickup elements may be provided on the camera head 5005. In this case, a plurality of relay optical systems are provided in the inside of the lens barrel 5003 in order to guide observation light to each of the plurality of image pickup elements.

(Various Apparatus Incorporated in Cart)

The CCU 5039 includes a central processing unit (CPU), a graphics processing unit (GPU) or the like and integrally controls operation of the endoscope 5001 and the display apparatus 5041. In particular, the CCU 5039 performs, for an image signal received from the camera head 5005, various image processes for displaying an image based on the image signal such as, for example, a development process (demosaic process). The CCU 5039 provides the image signal for which the image processes have been performed to the display apparatus 5041. Further, the CCU 5039 transmits a control signal to the camera head 5005 to control driving of the camera head 5005. The control signal may include information relating to an image pickup condition such as a magnification or a focal distance.

The display apparatus 5041 displays an image based on an image signal for which the image processes have been performed by the CCU 5039 under the control of the CCU 5039. If the endoscope 5001 is ready for imaging of a high resolution such as 4K (horizontal pixel number 3840×vertical pixel number 2160), 8K (horizontal pixel number 7680×vertical pixel number 4320) or the like and/or ready for 3D display, then a display apparatus by which corresponding display of the high resolution and/or 3D display are possible may be used as the display apparatus 5041. Where the apparatus is ready for imaging of a high resolution such as 4K or 8K, if the display apparatus used as the display apparatus 5041 has a size of equal to or not less than 55 inches, then a more immersive experience can be obtained. Further, a plurality of display apparatus 5041 having different resolutions and/or different sizes may be provided in accordance with purposes.

The light source apparatus 5043 includes a light source such as, for example, a light emitting diode (LED) and supplies irradiation light for imaging of a surgical region to the endoscope 5001.

The arm controlling apparatus 5045 includes a processor such as, for example, a CPU and operates in accordance with a predetermined program to control driving of the arm unit 5031 of the supporting arm apparatus 5027 in accordance with a predetermined controlling method.

An inputting apparatus 5047 is an input interface for the endoscopic surgery system 5000. A user can perform inputting of various kinds of information or instruction inputting to the endoscopic surgery system 5000 through the inputting apparatus 5047. For example, the user would input various kinds of information relating to surgery such as physical information of a patient, information regarding a surgical procedure of the surgery and so forth through the inputting apparatus 5047. Further, the user would input, for example, an instruction to drive the arm unit 5031, an instruction to change an image pickup condition (type of irradiation light, magnification, focal distance or the like) by the endoscope 5001, an instruction to drive the energy treatment tool 5021 or the like through the inputting apparatus 5047.

The type of the inputting apparatus 5047 is not limited and may be that of any one of various known inputting apparatus. As the inputting apparatus 5047, for example, a mouse, a keyboard, a touch panel, a switch, a foot switch 5057 and/or a lever or the like may be applied. Where a touch panel is used as the inputting apparatus 5047, it may be provided on the display face of the display apparatus 5041.

Otherwise, the inputting apparatus 5047 is a device to be mounted on a user such as, for example, a glasses type wearable device or a head mounted display (HMD), and various kinds of inputting are performed in response to a gesture or a line of sight of the user detected by any of the devices mentioned. Further, the inputting apparatus 5047 includes a camera which can detect a motion of a user, and various kinds of inputting are performed in response to a gesture or a line of sight of a user detected from a video imaged by the camera. Further, the inputting apparatus 5047 includes a microphone which can collect the voice of a user, and various kinds of inputting are performed by voice collected by the microphone. By configuring the inputting apparatus 5047 such that various kinds of information can be inputted in a contactless fashion in this manner, especially a user who belongs to a clean area (for example, the surgeon 5067) can operate an apparatus belonging to an unclean area in a contactless fashion. Further, since the user can operate an apparatus without releasing a possessed surgical tool from its hand, the convenience to the user is improved.

A treatment tool controlling apparatus 5049 controls driving of the energy treatment tool 5021 for cautery or incision of a tissue, sealing of a blood vessel or the like. A pneumoperitoneum apparatus 5051 feeds gas into a body lumen of the patient 5071 through the pneumoperitoneum tube 5019 to inflate the body lumen in order to secure the field of view of the endoscope 5001 and secure the working space for the surgeon. A recorder 5053 is an apparatus capable of recording various kinds of information relating to surgery. A printer 5055 is an apparatus capable of printing various kinds of information relating to surgery in various forms such as a text, an image or a graph.

In the following, especially a characteristic configuration of the endoscopic surgery system 5000 is described in more detail.

(Supporting Arm Apparatus)

The supporting arm apparatus 5027 includes the base unit 5029 serving as a base, and the arm unit 5031 extending from the base unit 5029. In the example depicted, the arm unit 5031 includes the plurality of joint portions 5033a, 5033b and 5033c and the plurality of links 5035a and 5035b connected to each other by the joint portion 5033b. In FIG. 1, for simplified illustration, the configuration of the arm unit 5031 is depicted in a simplified form. Actually, the shape, number and arrangement of the joint portions 5033a to 5033c and the links 5035a and 5035b and the direction and so forth of axes of rotation of the joint portions 5033a to 5033c can be set suitably such that the arm unit 5031 has a desired degree of freedom. For example, the arm unit 5031 may preferably be configured such that it has a degree of freedom equal to or not less than 6 degrees of freedom. This makes it possible to move the endoscope 5001 freely within the movable range of the arm unit 5031. Consequently, it becomes possible to insert the lens barrel 5003 of the endoscope 5001 from a desired direction into a body lumen of the patient 5071.

An actuator is provided in each of the joint portions 5033a to 5033c, and the joint portions 5033a to 5033c are configured such that they are rotatable around predetermined axes of rotation thereof by driving of the respective actuators. The driving of the actuators is controlled by the arm controlling apparatus 5045 to control the rotational angle of each of the joint portions 5033a to 5033c thereby to control driving of the arm unit 5031. Consequently, control of the position and the posture of the endoscope 5001 can be implemented. Thereupon, the arm controlling apparatus 5045 can control driving of the arm unit 5031 by various known controlling methods such as force control or position control.

For example, if the surgeon 5067 suitably performs operation inputting through the inputting apparatus 5047 (including the foot switch 5057), then driving of the arm unit 5031 may be controlled suitably by the arm controlling apparatus 5045 in response to the operation input to control the position and the posture of the endoscope 5001. After the endoscope 5001 at the distal end of the arm unit 5031 is moved from an arbitrary position to a different arbitrary position by the control just described, the endoscope 5001 can be supported fixedly at the position after the movement. It is to be noted that the arm unit 5031 may be operated in a master-slave fashion. In this case, the arm unit 5031 may be remotely controlled by the user through the inputting apparatus 5047 which is placed at a place remote from the surgery room.

Further, where force control is applied, the arm controlling apparatus 5045 may perform power-assisted control to drive the actuators of the joint portions 5033a to 5033c such that the arm unit 5031 may receive external force by the user and move smoothly following the external force. This makes it possible to move, when the user directly touches with and moves the arm unit 5031, the arm unit 5031 with comparatively weak force. Accordingly, it becomes possible for the user to move the endoscope 5001 more intuitively by a simpler and easier operation, and the convenience to the user can be improved.

Here, generally in endoscopic surgery, the endoscope 5001 is supported by a medical doctor called scopist. In contrast, where the supporting arm apparatus 5027 is used, the position of the endoscope 5001 can be fixed more certainly without hands, and therefore, an image of a surgical region can be obtained stably and surgery can be performed smoothly.

It is to be noted that the arm controlling apparatus 5045 may not necessarily be provided on the cart 5037. Further, the arm controlling apparatus 5045 may not necessarily be a single apparatus. For example, the arm controlling apparatus 5045 may be provided in each of the joint portions 5033a to 5033c of the arm unit 5031 of the supporting arm apparatus 5027 such that the plurality of arm controlling apparatus 5045 cooperate with each other to implement driving control of the arm unit 5031.

(Light Source Apparatus)

The light source apparatus 5043 supplies irradiation light upon imaging of a surgical region to the endoscope 5001. The light source apparatus 5043 includes a white light source which includes, for example, an LED, a laser light source or a combination of them. In this case, where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 5043. Further, in this case, if laser beams from the respective RGB laser light sources are irradiated time-divisionally on an observation target and driving of the image pickup elements of the camera head 5005 is controlled in synchronism with the irradiation timings, then images individually corresponding to the R, G and B colors can be picked up time-divisionally. According to the method just described, a color image can be obtained even if a color filter is not provided for the image pickup element.

Further, driving of the light source apparatus 5043 may be controlled such that the intensity of light to be outputted is changed for each predetermined time. By controlling driving of the image pickup element of the camera head 5005 in synchronism with the timing of the change of the intensity of light to acquire images time-divisionally and synthesizing the images, an image of a high dynamic range free from underexposed blocked up shadows and overexposed highlights can be created.

Further, the light source apparatus 5043 may be configured to supply light of a predetermined wavelength band ready for special light observation. In special light observation, for example, by utilizing the wavelength dependency of absorption of light in a body tissue to irradiate light of a narrower band in comparison with irradiation light upon ordinary observation (namely, white light), narrow band light observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 5043 can be configured to supply such narrowband light and/or excitation light suitable for special light observation as described above.

(Camera Head and CCU)

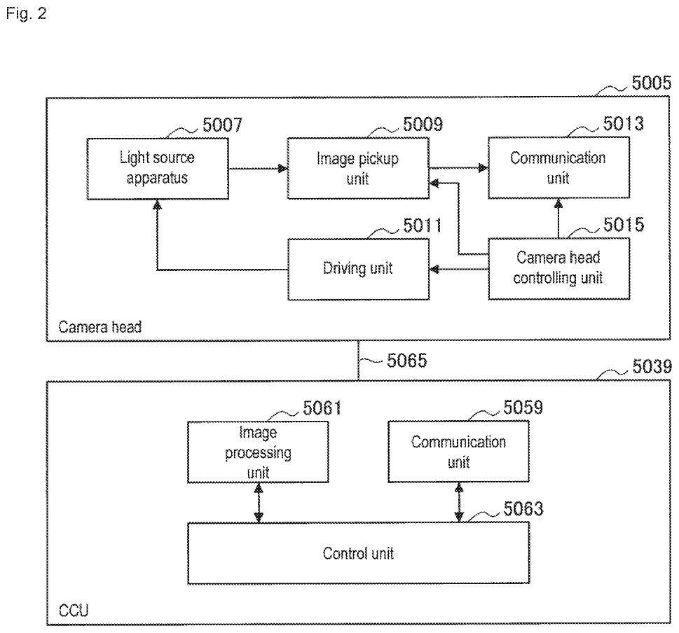

Functions of the camera head 5005 of the endoscope 5001 and the CCU 5039 are described in more detail with reference to FIG. 2. FIG. 2 is a block diagram depicting an example of a functional configuration of the camera head 5005 and the CCU 5039 depicted in FIG. 1.

Referring to FIG. 2, the camera head 5005 has, as functions thereof, a lens unit 5007, an image pickup unit 5009, a driving unit 5011, a communication unit 5013 and a camera head controlling unit 5015. Further, the CCU 5039 has, as functions thereof, a communication unit 5059, an image processing unit 5061 and a control unit 5063. The camera head 5005 and the CCU 5039 are connected to be bidirectionally communicable to each other by a transmission cable 5065.

First, a functional configuration of the camera head 5005 is described. The lens unit 5007 is an optical system provided at a connecting location of the camera head 5005 to the lens barrel 5003. Observation light taken in from a distal end of the lens barrel 5003 is introduced into the camera head 5005 and enters the lens unit 5007. The lens unit 5007 includes a combination of a plurality of lenses including a zoom lens and a focusing lens. The lens unit 5007 has optical properties adjusted such that the observation light is condensed on a light receiving face of the image pickup element of the image pickup unit 5009. Further, the zoom lens and the focusing lens are configured such that the positions thereof on their optical axis are movable for adjustment of the magnification and the focal point of a picked up image.

The image pickup unit 5009 includes an image pickup element and disposed at a succeeding stage to the lens unit 5007. Observation light having passed through the lens unit 5007 is condensed on the light receiving face of the image pickup element, and an image signal corresponding to the observation image is generated by photoelectric conversion of the image pickup element. The image signal generated by the image pickup unit 5009 is provided to the communication unit 5013.

As the image pickup element which is included by the image pickup unit 5009, an image sensor, for example, of the complementary metal oxide semiconductor (CMOS) type is used which has a Bayer array and is capable of picking up an image in color. It is to be noted that, as the image pickup element, an image pickup element may be used which is ready, for example, for imaging of an image of a high resolution equal to or not less than 4K. If an image of a surgical region is obtained in a high resolution, then the surgeon 5067 can comprehend a state of the surgical region in enhanced details and can proceed with the surgery more smoothly.

Further, the image pickup element which is included by the image pickup unit 5009 includes such that it has a pair of image pickup elements for acquiring image signals for the right eye and the left eye compatible with 3D display. Where 3D display is applied, the surgeon 5067 can comprehend the depth of a living body tissue in the surgical region more accurately. It is to be noted that, if the image pickup unit 5009 is configured as that of the multi-plate type, then a plurality of systems of lens units 5007 are provided corresponding to the individual image pickup elements of the image pickup unit 5009.

The image pickup unit 5009 may not necessarily be provided on the camera head 5005. For example, the image pickup unit 5009 may be provided just behind the objective lens in the inside of the lens barrel 5003.

The driving unit 5011 includes an actuator and moves the zoom lens and the focusing lens of the lens unit 5007 by a predetermined distance along the optical axis under the control of the camera head controlling unit 5015. Consequently, the magnification and the focal point of a picked up image by the image pickup unit 5009 can be adjusted suitably.

The communication unit 5013 includes a communication apparatus for transmitting and receiving various kinds of information to and from the CCU 5039. The communication unit 5013 transmits an image signal acquired from the image pickup unit 5009 as RAW data to the CCU 5039 through the transmission cable 5065. Thereupon, in order to display a picked up image of a surgical region in low latency, preferably the image signal is transmitted by optical communication. This is because, upon surgery, the surgeon 5067 performs surgery while observing the state of an affected area through a picked up image, it is demanded for a moving image of the surgical region to be displayed on the real time basis as far as possible in order to achieve surgery with a higher degree of safety and certainty. Where optical communication is applied, a photoelectric conversion module for converting an electric signal into an optical signal is provided in the communication unit 5013. After the image signal is converted into an optical signal by the photoelectric conversion module, it is transmitted to the CCU 5039 through the transmission cable 5065.

Further, the communication unit 5013 receives a control signal for controlling driving of the camera head 5005 from the CCU 5039. The control signal includes information relating to image pickup conditions such as, for example, information that a frame rate of a picked up image is designated, information that an exposure value upon image picking up is designated and/or information that a magnification and a focal point of a picked up image are designated. The communication unit 5013 provides the received control signal to the camera head controlling unit 5015. It is to be noted that also the control signal from the CCU 5039 may be transmitted by optical communication. In this case, a photoelectric conversion module for converting an optical signal into an electric signal is provided in the communication unit 5013. After the control signal is converted into an electric signal by the photoelectric conversion module, it is provided to the camera head controlling unit 5015.

It is to be noted that the image pickup conditions such as the frame rate, exposure value, magnification or focal point are set automatically by the control unit 5063 of the CCU 5039 on the basis of an acquired image signal. In other words, an auto exposure (AE) function, an auto focus (AF) function and an auto white balance (AWB) function are incorporated in the endoscope 5001.

The camera head controlling unit 5015 controls driving of the camera head 5005 on the basis of a control signal from the CCU 5039 received through the communication unit 5013. For example, the camera head controlling unit 5015 controls driving of the image pickup element of the image pickup unit 5009 on the basis of information that a frame rate of a picked up image is designated and/or information that an exposure value upon image picking up is designated. Further, for example, the camera head controlling unit 5015 controls the driving unit 5011 to suitably move the zoom lens and the focus lens of the lens unit 5007 on the basis of information that a magnification and a focal point of a picked up image are designated. The camera head controlling unit 5015 may further include a function for storing information for identifying the lens barrel 5003 and/or the camera head 5005.

It is to be noted that, by disposing the components such as the lens unit 5007 and the image pickup unit 5009 in a sealed structure having high airtightness and waterproof, the camera head 5005 can be provided with resistance to an autoclave sterilization process.

Now, a functional configuration of the CCU 5039 is described. The communication unit 5059 includes a communication apparatus for transmitting and receiving various kinds of information to and from the camera head 5005. The communication unit 5059 receives an image signal transmitted thereto from the camera head 5005 through the transmission cable 5065. Thereupon, the image signal may be transmitted preferably by optical communication as described above. In this case, for the compatibility with optical communication, the communication unit 5059 includes a photoelectric conversion module for converting an optical signal into an electric signal. The communication unit 5059 provides the image signal after conversion into an electric signal to the image processing unit 5061.

Further, the communication unit 5059 transmits, to the camera head 5005, a control signal for controlling driving of the camera head 5005. The control signal may also be transmitted by optical communication.

The image processing unit 5061 performs various image processes for an image signal in the form of RAW data transmitted thereto from the camera head 5005. The image processes include various known signal processes such as, for example, a development process, an image quality improving process (a bandwidth enhancement process, a super-resolution process, a noise reduction (NR) process and/or an image stabilization process) and/or an enlargement process (electronic zooming process). Further, the image processing unit 5061 performs a detection process for an image signal in order to perform AE, AF and AWB.

The image processing unit 5061 includes a processor such as a CPU or a GPU, and when the processor operates in accordance with a predetermined program, the image processes and the detection process described above can be performed. It is to be noted that, where the image processing unit 5061 includes a plurality of GPUs, the image processing unit 5061 suitably divides information relating to an image signal such that image processes are performed in parallel by the plurality of GPUs.

The control unit 5063 performs various kinds of control relating to image picking up of a surgical region by the endoscope 5001 and display of the picked up image. For example, the control unit 5063 generates a control signal for controlling driving of the camera head 5005. Thereupon, if image pickup conditions are inputted by the user, then the control unit 5063 generates a control signal on the basis of the input by the user. Alternatively, where the endoscope 5001 has an AE function, an AF function and an AWB function incorporated therein, the control unit 5063 suitably calculates an optimum exposure value, focal distance and white balance in response to a result of a detection process by the image processing unit 5061 and generates a control signal.

Further, the control unit 5063 controls the display apparatus 5041 to display an image of a surgical region on the basis of an image signal for which image processes have been performed by the image processing unit 5061. Thereupon, the control unit 5063 recognizes various objects in the surgical region image using various image recognition technologies. For example, the control unit 5063 can recognize a surgical tool such as forceps, a particular living body region, bleeding, mist when the energy treatment tool 5021 is used and so forth by detecting the shape, color and so forth of edges of the objects included in the surgical region image. The control unit 5063 causes, when it controls the display unit 5041 to display a surgical region image, various kinds of surgery supporting information to be displayed in an overlapping manner with an image of the surgical region using a result of the recognition. Where surgery supporting information is displayed in an overlapping manner and presented to the surgeon 5067, the surgeon 5067 can proceed with the surgery more safety and certainty.

The transmission cable 5065 which connects the camera head 5005 and the CCU 5039 to each other is an electric signal cable ready for communication of an electric signal, an optical fiber ready for optical communication or a composite cable ready for both of electrical and optical communication.

Here, while, in the example depicted, communication is performed by wired communication using the transmission cable 5065, the communication between the camera head 5005 and the CCU 5039 may be performed otherwise by wireless communication. Where the communication between the camera head 5005 and the CCU 5039 is performed by wireless communication, there is no necessity to lay the transmission cable 5065 in the surgery room. Therefore, such a situation that movement of medical staff in the surgery room is disturbed by the transmission cable 5065 can be eliminated.

An example of the endoscopic surgery system 5000 to which the technology according to an embodiment of the present disclosure can be applied has been described above. It is to be noted here that, although the endoscopic surgery system 5000 has been described as an example, the system to which the technology according to an embodiment of the present disclosure can be applied is not limited to the example. For example, the technology according to an embodiment of the present disclosure may be applied to a soft endoscopic system for inspection or a microscopic surgery system.

The technology according to an embodiment of the present disclosure can be applied suitably to the control unit 5063 from among the components described hereinabove. Specifically, the technology according to an embodiment of the present disclosure relates to endoscopy and/or microscopy or any kind of medical imaging. By applying the technology according to an embodiment of the present disclosure to the endoscopy and/or microscopy technology and/or medical imaging more generally, the depth of the vasculature can be more accurately and easily found. This reduces the likelihood of injury or death of a patient and improves the efficiency with which the medical procedure (such as surgery) can be carried out.

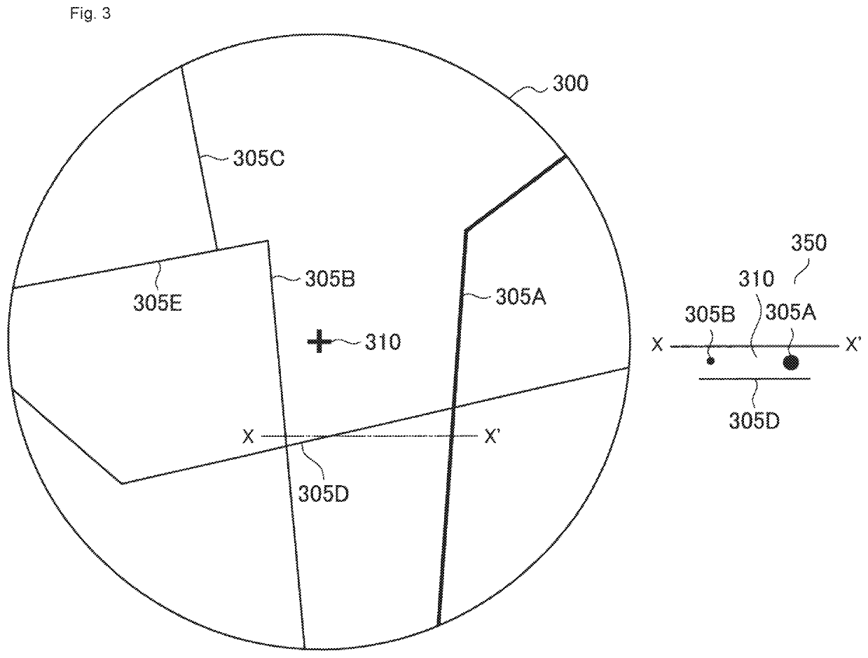

Referring to FIG. 3, an embodiment of the disclosure is shown. Specifically, endoscope view 300 shows an image captured by endoscope 5001. Within the endoscope view 300 is vasculature 305A-305E. The vasculature is an example of a vessel carrying fluid around the body. In the example of FIG. 3, the vasculature 305A-305E carries blood through the body. The vasculature is usually located within tissue at various depths. Accordingly, if the depth of the vasculature is not accurately determined during the surgical procedure, there is a likelihood that the vasculature may be damaged by, especially, invasive procedures.

As the skilled person would appreciate, the vessels which compose the vasculature 305A-305E are of varying lengths, directions and diameters. Additionally, the vessels within vasculature 305A-305E reside at various depths within tissue 310 and are of varying diameters. Additionally shown in endoscope view 300 is the centre of the view 310. This is shown in endoscope view 300 as a “+” sign. The purpose of the centre of view 310 is to be a known position within the endoscope view 300 from which the various positions of the vessels within the vasculature 305A-305E maybe referenced. Accordingly, the centre of view 310 may be considered to be a reference point and may be located elsewhere within the endoscope view 300.

Additionally shown in FIG. 3 is a cross section view 350. Specifically, the cross section 350 along the line X-X′ is shown in FIG. 3.

Referring to the cross section view 350, the tissue 310 includes a first vessel 305A, a second vessel 305B and a third vessel 305D as can be seen from the cross section view 350, the diameter of the first vessel 305A is larger than the diameter of the second vessel 305B. It will be apparent that as the third vessel 305D runs along the length of cross section X-X′, the diameter of the third vessel 305D cannot be determined. However, as will be apparent, the position of the third vessel 305D is below that of the first vessel 305A and the second vessel 305B. In other words, the third vessel 305D is located deeper within the tissue 310 than both the first vessel 305A and the second vessel 305B. This means that the third vessel 305D is located underneath the first vessel 305A and the second vessel 305B. This depth is determined using embodiments of the present disclosure.

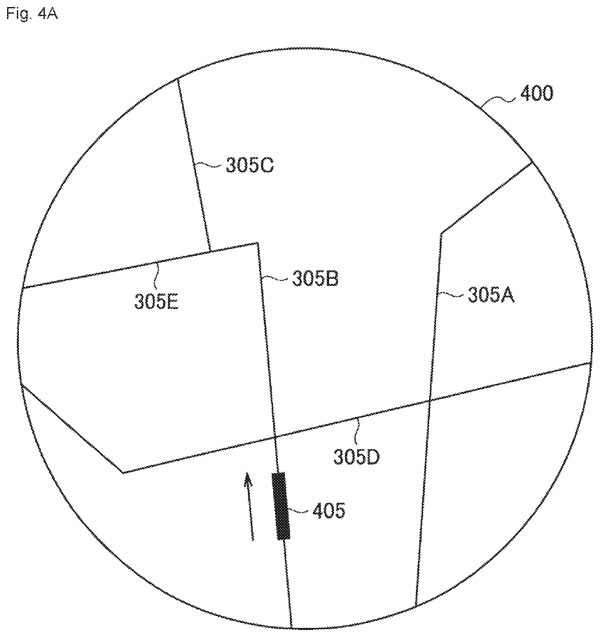

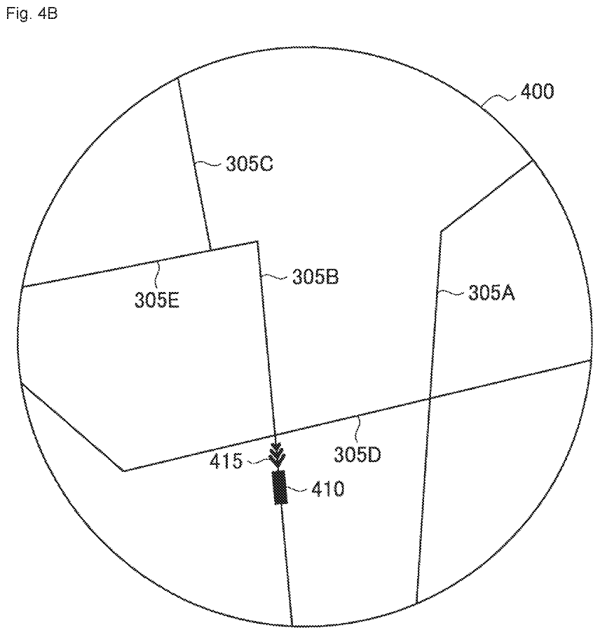

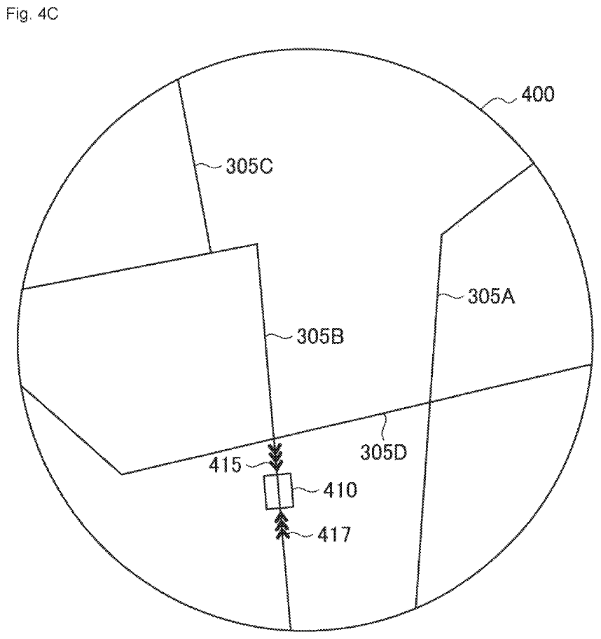

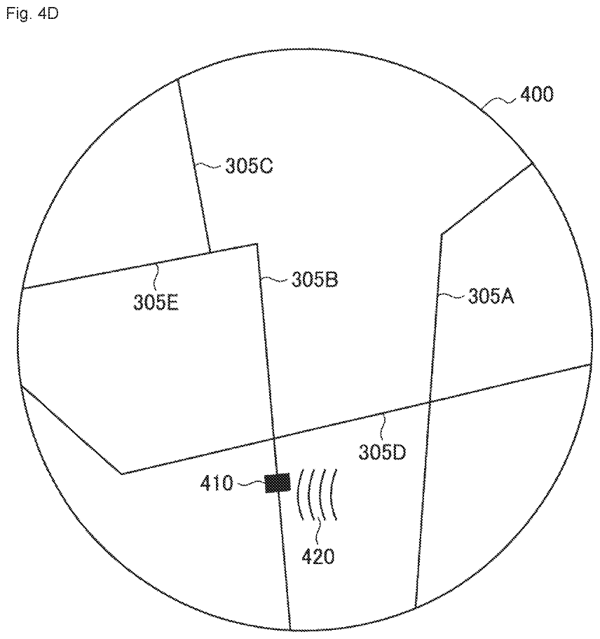

During surgery, the patient's heart will beat. This sends a pulse of blood through the vasculature shown in endoscope view 300. This occurrence is shown in FIG. 4A.

Specifically, FIG. 4A shows an endoscope view 400 which includes the vasculature 305A-305E as shown in FIG. 3. Additionally, a pulse of blood 405 is shown in endoscope view 400. The direction of travel of the pulse of blood is indicated by an arrow in FIG. 4A. As can be seen from FIG. 4A, the pulse of blood 405 captured in endoscope view 400 is travelling through the second vessel 305B. As will be appreciated, the pulse of blood 405 will arrive in endoscope view 400 a period of time after the heart-beat of the patient. This delay between the heart pumping and the pulse of blood 405 arriving in endoscope view 400 can be determined by measuring the time difference between the beat of the patient's heart, measured by an electrocardiogram (ECG) and the arrival of the pulse of blood 405 into the endoscope view 400. This time difference information is useful to determine when the pulse of blood arrives in the second vessel 305B.

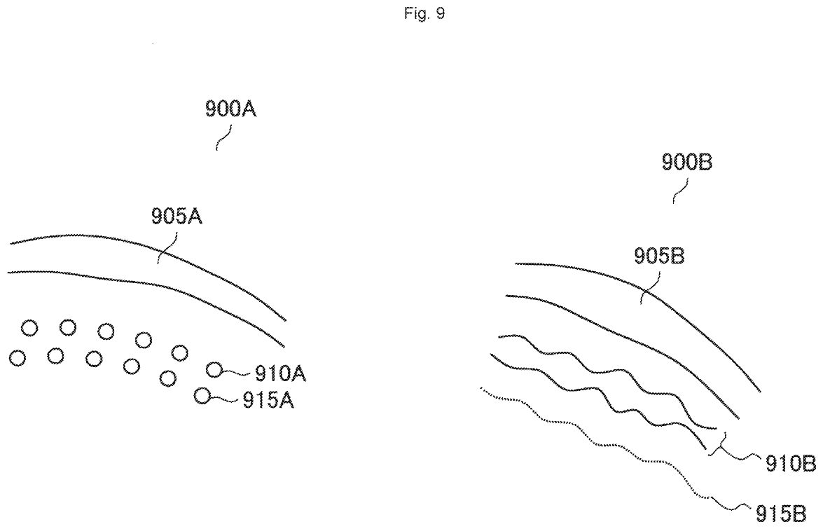

Referring to FIG. 4B, the endoscope view 400 according to embodiments of the disclosure is shown. In the endoscope view 400 of FIG. 4B, a modified pulse of blood 410 is shown. The modified pulse of blood results from the pulse of blood 405 in FIG. 4A having a flow modifying pulse 415 applied to it. In embodiments of the disclosure, the flow modifying pulse 415 may be a photo acoustic force within the second blood vessel 305B. This may be generated using a pulse laser. The mechanism for generating the photo acoustic force will be explained later with reference to FIG. 9.

The purpose of the flow modifying pulse 415 is to apply a fixed force amplitude to the flow of blood to modify the flow of blood. In this instance, the flow modifying pulse 415 is in the direction opposing the direction of the flow of blood. By opposing the pulse of blood, the movement of the pulse of blood through the second vessel 305B is reduced meaning that the diameter of the second vessel 305B increases due to the increased volume of blood at the point at which the flow is modified. This increases the stiffness the vessel so that its elasticity is similar to that of the surrounding tissue. This improves the signal to noise ratio of a depth analysis using either a surface acoustic wave (SAW) and/or more accurate depth resolution of vessels using laser speckle imaging. Whilst this is a desirable effect on its own, this more accurate depth measurement, allows better estimation of flow volume and vessels sizes which is important for diagnostic and treatment planning applications such as the identification of hypertension within the patient. This is achieved by applying a photoacoustic signal (the flow modifying pulse 415), to increase the diameter of the vessel. This increase in diameter also increases the stiffness of the vessel relative to the surrounding tissue. Accordingly, the depth sensitivity will be improved. Of course, the skilled person will appreciate that the flow modifying pulse 415 may not be required if the depth measurement occurs at the point during which the pulse of blood 405 in FIG. 4A passes through the second vessel 305B. This will be explained later. In other words, the skilled person will appreciate that when the pulse of blood flows through the vessel, the diameter of the vessel will naturally increase, thus giving enhanced stiffness.

Referring to FIG. 4C, a further embodiment of the discussion of FIG. 4B is shown. In this further embodiment, the endoscope view 400 shows a further flow modifying pulse 417 being applied to the modified pulse of blood 410. In the example of FIG. 4C, the further flow modifying pulse 417 is applied behind the modified pulse of blood 410 in the direction of travel of the pulse. In other words, the modified pulse of blood 410 is effectively squashed between the flow modifying pulse 415 and the further flow modifying pulse 417. Both the flow modifying pulse 415 and the further flow modifying pulse 417 act in opposite directions at opposite sides to the modified pulse of blood 410 to squash the modified pulse of blood even further than the embodiment of FIG. 4B. This further flow modifying pulse 417 being applied behind the modified pulse of blood 410 and in a direction opposite to the flow modifying pulse 415 has the effect of further enhancing the stiffness contrast of the vessels. This, in turn, improves the signal to noise ratio of the embodiment of FIG. 4B.

Although the forgoing describes application of the further flow modifying pulse 417 in a direction opposite to the flow modifying pulse 415, the disclosure is not so limited. In this further embodiment, the further flow modifying pulse 417 may be applied in any direction, even the same or similar direction to the flow modifying pulse 415. In particular, if the stiffness contrast of one side of the vessel is required, the further flow modifying pulse 417 may be applied in the direction of the side requiring enhancement. For example, in FIG. 4C, if the right hand side of the modified pulse of blood 410 requires further enhancement, the further flow modifying pulse 417 may be located on the left side of the modified pulse of blood 410 facing in the right direction. Additionally, or alternatively, the further flow modifying pulse 417 may be additive to the flow modifying pulse 415 such that the totality of the flow modifying pulse applied to the modified pulse of blood 410 stops the modified pulse of blood 410 progressing through the second vessel 305B. This will provide even further enhanced stiffness contrast of the second vessel 305B.

Referring to FIG. 4D, after the application of the flow modifying pulse 410 and optionally the further flow modifying pulse 417, a Surface Acoustic Wave (SAW) wave is applied to the modified pulse of blood 410. A discussion of SAW waves is provided under the heading “Surface Acoustic Waves (SAWs)” (below). As noted earlier, the purpose of the SAW wave is to determine the depth of the second vessel 305B. Therefore, although FIG. 4D shows the SAW wave being applied to the modified pulse of blood that has the enhanced stiffness contrast, the disclosure is not so limited. Indeed, the SAW wave may be applied to the pulse of blood 405 as this has enhanced stiffness compared with the second vessel 305B with no blood flowing therethrough. In other words, when the pulse of blood passes through the second vessel 305B following a heart-beat, the diameter of the second vessel 305B increases to allow the blood to pass. This enhances the stiffness of the second vessel 305B without applying the flow modifying pulse 417. The SAW wave may then be applied to the second vessel 305B at the time of enhanced stiffness caused by the pulse of blood.

As the measurements of SAW propagation is a known technique to determine the depth and elasticity of different layers having distinct mechanical properties is known, this will not be explained in any detail hereinafter.

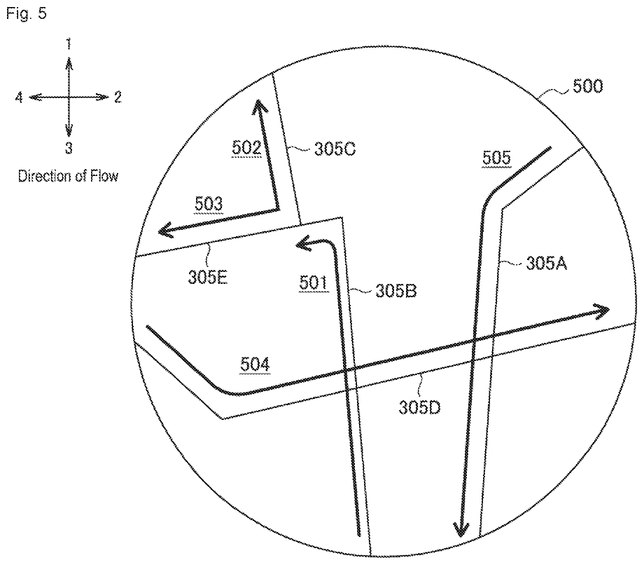

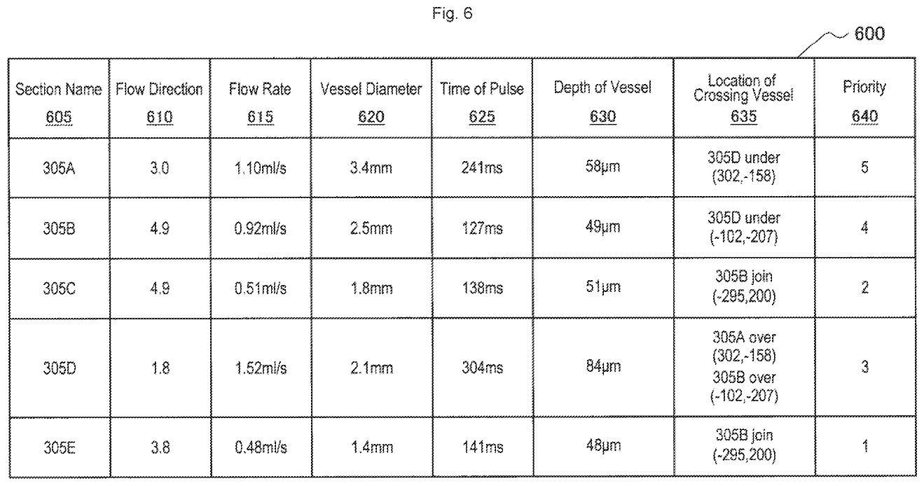

Referring to FIG. 6, a data structure 600 is shown. Embodiments of the disclosure, the data structure 600 may be of the form of a table or a database or the like. The data structure 600 used within the CCU 5039 and is stored within a storage medium (not shown). The data structure 600 will now be explained with reference to FIG. 5. As will be apparent the endoscope view 500 from FIG. 5 includes the vasculature of FIGS. 4A-4D. Each section of the vasculature is identified within data structure 600. In the examples of data structure 600, the vasculature is segmented. Specifically each segment of the vasculature is given a unique identifier. In the example of data structure 600, the segments are given the unique identifier 305A-305E. Although the diagram indicates that the entire vessel segment is given the unique identifier, in reality, each segment may be broken down into further segments or the unique identifier will be attributed to a small portion of a particular section. This allows for branching of various segments and allows for varying flow volume of blood passing through a particular segment. In this regard, therefore, it is envisaged that a particular point within the length of the section will be attributed with the identifier. This particular point may be a mid-point along the length of the section or the like.

The data structure 600 also includes the direction of flow associated with each vessel segment. This provided in column 610.

From FIG. 5 it is seen that there are arrows numbered 1-4 in the top left hand corner of FIG. 5. These arrows indicate the nomenclature used in column 610 of the data structure 600 in identifying the flow direction through a particular vessel segment. In order to illustrate the nomenclature, the blood flow through each respective segment within the endoscope view 500 is provided in the diagram as solid line arrows 501-505.

In FIG. 5, the blood flow through the first vessel 305A is vertically downward.

This means that in the nomenclature of FIG. 5, the blood flow is in the direction 3.0. The direction of the blood flow through the second vessel 305B is upward with a slight angle to the left. This means that in the nomenclature of FIG. 5, the blood flow through the second vessel 305B is in the direction 4.9.

The remaining flow directions in column 610 follow this nomenclature as will be apparent to the skilled person.

In order to determine the direction and velocity of blood flow, the cross correlation of the Laser Speckle Intensity (see heading “Laser Speckle Contrast Imaging (LSCI)”) from two points within the identified blood vessel are used. This is achieved by applying a pixel amplitude threshold and noise filtering to the speckle image and then pixel locations are used to define the relative vessel dimensions and two dimensional locations within the captured image. This would be apparent to the skilled person.

Returning to FIG. 6, column 615 stores the velocity of the blood flow determined using the LSCI technique through the relevant vessel section. Column 620 stores the vessel diameter of each vessel segment. The vessel diameter can be determined using a pre-acquired image scale as is known. The flow rate and the vessel diameter are used to determine the flow volume of each vessel segment. Specifically, the flow volume of each vessel segment is calculated as a function of the vessel diameter and the flow rate. The flow volume of each vessel segment is calculated as a function of vessel diameter and the flow velocity using the equation (1) below.

$$\begin{matrix}[ Math . 1 ] & \\ Q = v \cdot π {( \frac{d}{2} )}^{2} & Equation ( 1 )\end{matrix}$$

Herein, d is the diameter of the vessel, v is the flow velocity and Q is the flow volume.

In column 625, the time of the pulse is stored. This is the time difference between the patient's heartbeat measured by an electrocardiogram (ECG) and the time at which the pulse of blood passes through the vessel. The pulse of blood is identified because there is a change in the diameter of the vessel. This change in diameter is observed over a period of time (for example, 10 heartbeats) and an average time of the time of pulse is stored. Using this, for example, the pulse of blood passing through the first vessel section 305A is measured at 241 milliseconds after the heartbeat.

The depth of vessel column 630 is completed when methods according to embodiments of the disclosure are performed. This will be explained later.

Additionally, once the depth of vessel column 630 is completed, the location of crossing vessel 635 is completed. In order to determine the location of the crossing vessel, the intersection of each vessel with each other vessel in the vasculature is derived using object recognition. For example, the path of each vessel is traversed and where the vessel intersects with another vessel, the location of the intersection is defined. This location is a pixel co-ordinate relative to the centre of the view 310.

Once the depth of each vessel is determined, using embodiments of the disclosure, the location and whether the vessel passes over or under the intersecting is completed.

Finally, a priority column 640 is provided. The priority column provides the order in which the vessel segments 305A-305E have the blood flow modified. In one embodiment, the order may be defined such that the depth of the vessels having the smallest diameter is performed first and the order in which the depths are determined is performed in increasing diameter. This is the case with the embodiment of FIG. 6. This order selection is useful because the vessels that would benefit most from active flow modification (i.e. the smaller diameter vessels) are performed first.

Of course, the disclosure is not limited to this and the order in which the depth of the vessel is determined may also be chosen to prevent interference from one section to the next. In this case, consecutive flow modification may be applied to vessels located further than a predetermined distance from one another. Other types of ordering, such applying the flow modification to the vessels having the largest diameter vessels first is also envisaged. This ordering may be appropriate when the vessels having higher blood flow volume are to be modified first. Of course, other factors such as the flow rate or even the flow direction may determine the order in which the vessels are analysed. For example, all vessels having blood flow in the same direction may be analysed first.

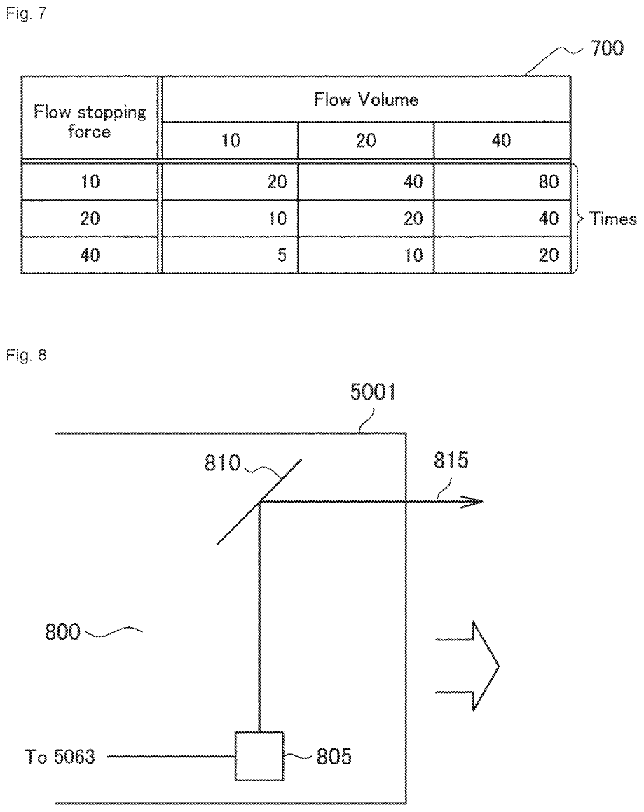

Referring to FIG. 7, a flow lookup table 700 is shown. The flow lookup table 700 shows the timings required to reach a maximum flow reduction given the force capability of the flow modification. In other words, it takes a predictable length of time to reach peak speed reduction for a given vessel target and flow modification force. If it is assumed that the coverage of the vessel lumen by the flow stopping force is efficient, the flow stopping lookup table 700 would consist of vessel flow volumes (flow velocity multiplied by vessel diameter) and known times to reach maximum flow reduction for a given flow modification force. The times for each the maximum flow reduction are achieved are shown in the lookup table 700.

Referring to FIG. 8, an endoscope 5001 according to embodiments of the disclosure is shown. The endoscope tip comprises a wave generation unit 800 according to embodiments of the disclosure. The wave generation unit 800 comprises a laser light source 805 that may be a solid state laser or equivalent. For example, the solid state laser may be a Vertical Cavity Surface Emitting Laser, or may be a laser provided over an optical fibre. In this case, the laser light source is located in the head of the endoscope or is an isolated laser light source provided in the medical imaging system. The optical fibre then carries the laser light to the appropriate location. The laser light source 805 is connected to the control unit 563 and is controlled by the control unit 5063. The wave generation unit 800 also includes a two axis microelectromechanical mirror (MEMs) mirror. The direction of the MEMs mirror is also controlled by the control unit 5063. The laser light source 805 fires the laser onto the two axis MEMs mirror 810 and the laser light is reflected in the direction indicated by the arrow. The laser light 815 from the laser light source 805 is then imparted onto the tissue 310. By applying a burst of laser light onto the tissue, longitudinal and transverse waves are provided through the tissue 310 using a known “Photoacoustic Technique” described below. It should be noted that the wave generating unit 800 may produce one or both of the flow modifying pulse or the SAW wave generation.

Of course, although the embodiment discussed in respect of FIG. 8 shows the laser light source 805 located in the endoscope 5001, the disclosure is not so limited. In particular, the laser light source 805 may be located in the head of the endoscope and an optical fibre may impart the laser light onto the two axis MEMs mirror 810.

Referring to FIG. 9, the mechanism for performing the photoacoustic technique is shown. In a first view 900A, the blood vessel 905A is shown. The blood in a first row 910A will have a wavefront generated first when the laser is imparted onto the tissue 310. The blood in a second row 915A cancels the wavefront from the first row 910A in a direction away from the blood vessel. In other words, the laser light energy is passed from the blood in the first row 910A to the blood in the second row 915A. Therefore, the wavefront is generated in parallel to the blood vessel 905A and crosses the vessel.

This is shown in a second view 900B, where a blood vessel 905B having the wavefront from the first row and second row (shown collectively as 910B) being cancelled by destructive interference 915B.

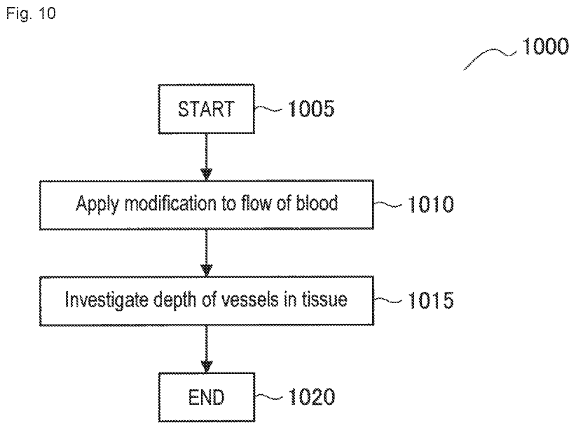

Referring to FIG. 10, a flowchart 1000 describing embodiments of the disclosure is shown. The process starts at step 1005. The process then moves to step 1010 where flow modifying pulse and optionally the further flow modifying pulse 417 is applied to the pulse of blood. The timing of the application of the flow modifying pulse 415 and the further flow modifying pulse 417 is defined in column 625. Specifically, the flow modifying pulse is applied at the time indicated in column 625 after the ECG detects the beat of the heart.

The order in which the modifications are applied is given in the priority column 640.

In the specific example of FIG. 6, the modification to the flow of blood is applied to section 305E first. This is because this vessel has the smallest vessel diameter. The location of the modification will be to oppose the direction of the blood flow. The flow of blood is given by the flow direction column 610 in the table of FIG. 6. In other words, as the blood vessel 305E travels in the direction 3.8, the modification to the flow of blood will be applied in the direction of 1.8 as this is substantially opposite to the flow of the blood.

The force of the flow modifier and the time for which the flow modification will be applied is provided in the lookup table of FIG. 7. Specifically, for a given flow volume (which is calculated as a function of the vessel diameter and the flow rate as in Equation 1) an appropriate flow stopping force is applied for a specified period of time. The flow stopping force and the time for which that flow stopping force needs to be applied is determined in advance via experimentation.

In the above example, the application of the flow modifying pulse 415 is performed in synchronisation with the natural pulse. However, the disclosure is not so limited. As explained above, the flow modification pulse is optional as the SAW may be applied as the heart is beating. Optionally, further flow modifications can also be included in the flow modification step 1010. This would cut the flow into an upstream branch which supplies an area outside the current section. This would increase blood pressure in the current area under investigation.

After the flow modifying pulse 415 and optionally the further modifying pulse 417 has been applied to the vessel under test (in this case vessel 305E) the depth of the vessels in the tissue is investigated in step 1015.

In order to achieve this, the surface acoustic wave is applied to the current vessel section under test. For example, multiple interfering SAWs are generated at a fixed, pre-defined distance from the vessel edge to create a wave that crosses the vessel perpendicular to its axis at all points. This is shown diagrammatically in FIG. 9.

It is possible that multiple, identical but temporally separated SAW waves are applied to the vessel under test. This is a known technique which allows the average phase velocity of different frequency components of the SAW wave to be measured. Of course, the disclosure is not so limited and optionally, given known SAW group velocity in soft tissue and the location at which the SAW wave is applied, the SAW initiating timing is chosen so that the SAW arrives at the vessel after maximum flow modification effect has been achieved. In other words, the SAW wave can be sent through the tissue so that the time at which the SAW wave interacts with the vessel under test coincides with the time at which the flow modifying pulse reduces the flow volume to a minimum.

Further, a single SAW wave may be applied to the vessel under test and the motion of the SAW wave as it passes across the vessel under test may be captured in two or more images. In this case, the average phase velocities of different frequency components of the wave can be determined by comparing the spatial frequency distribution within the SAW between two images of the same wave captured at different times. The average phase velocities of the wave as it crosses the blood vessel under test then are compared to a control measurement with the same propagation distance either before or after the blood vessel to determine the change in the SAW wave form as a result of the vessel. This allows the vessel depth to be determined using the affected frequency components and their known wavelength within the soft tissue.

In order to identify the SAWs within the image data, it is possible to apply a pixel amplitude threshold to the image data and subtract the known locations of the blood vessels in the vascular data. Optionally, other analysis functions such as shape/waveform recognition may be used to improve the SAW detection.

The SAW attributes such as the phase velocity are then determined. In order to determine the SAW attributes, the SAW wave form is measured by analysing the SAW in a one dimensional line perpendicular to the direction of propagation. For example, in the case of a laser speckled image, the intensity distribution along that one dimensional line is used. The frequency composition of the wave form in several windows along the one dimensional line is identified using the Fourier transform of the recorded waveform. The spatial distribution of different frequency components is then determined. The location of the measurements within the investigated area is then recorded. From this, the average phase velocity is determined and the depth of the vessel at a particular point is then determined. The column 630 in FIG. 6 is then populated for vessel section 305E. The process for determining the depth of the vessel section 305E then ends in step 1020.

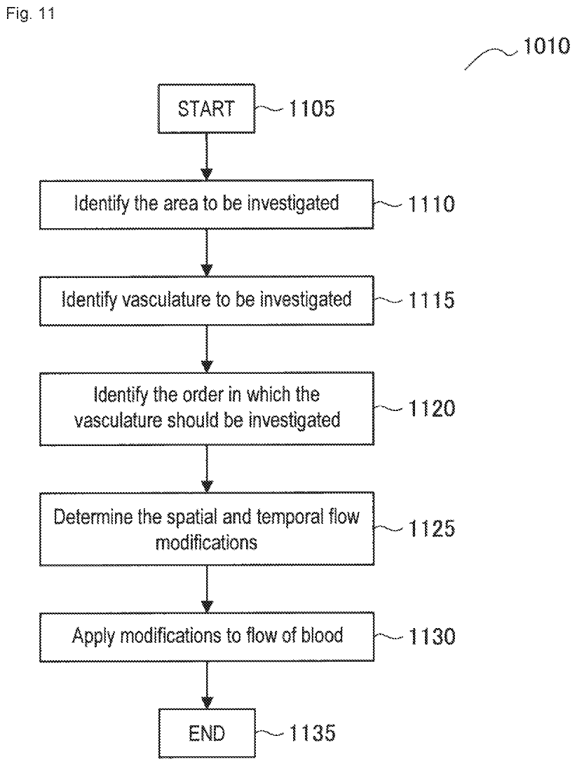

Referring to FIG. 11, a flowchart 1010 explaining the application of the flow modifying pulse is explained. The process steps at step 1105. The process moves to step 1110 which identifies the area to be investigated. This is the endoscope view of FIGS. 4A-4D and FIG. 5. Using object recognition, the vasculature within the area to be investigated is identified in step 1115. The process then moves to step 1120 where the order in which the vasculature should be investigated is identified. This information is taking from the priority column 640. The process then moves to step 1125 where the flow stopping force, the direction and the period of time for which the flow modifying pulse 415 and optionally the further flow modifying pulse 417 are to be applied is determined. The process then moves to step 1130 where the flow modifying pulse 415 is applied to the tissue 310. The process then ends at step 1135.

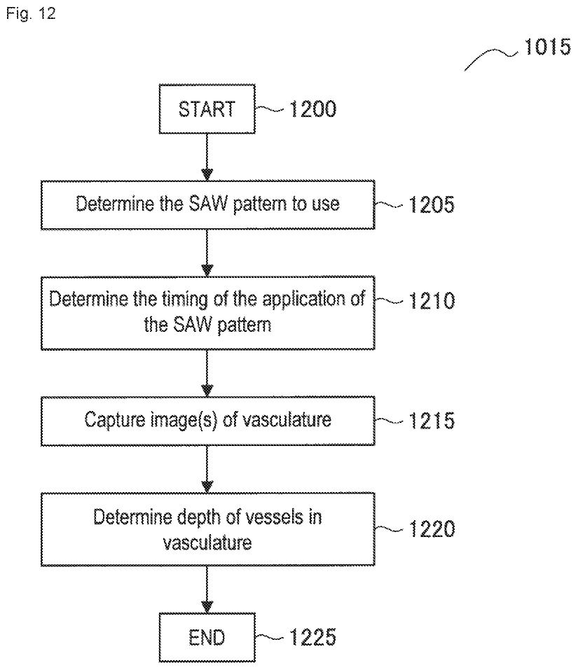

Referring to FIG. 12, the investigation of the depth of the vessels in the tissue is further explained. This process starts at step 1200. The process then moves to step 1205 where the SAW pattern to use is determined. This may be multiple, identical but temporally separated SAW waves, or may be a single SAW wave to be applied. After the SAW pattern is determined, the process moves to step 1210 where the timing of the application of the SAW pattern is determined. This is achieved by using the knowledge of the SAW group velocity and soft tissue and the times from the data structure 700. The SAW pattern is then applied to the tissue. The process moves to step 1215 where an image of the vasculature with the SAW applied is captured. Of course, if only a single SAW wave is applied then two or more images are captured showing the SAW wave passing through the vessel under test. The process then moves to step 1220 where the depth of vessels in the tissue is determined using the average phase velocity of different frequency components of the wave. The process then ends in step 1225.

Although the above disclosure relates to applying a flow modifying pulse 415, and optionally a further flow modifying pulse 417, the disclosure is not so limited. In some embodiments, the SAW wave may be used to modulate the blood flow eliminating the need for the flow modifying pulse 415. This may be through the generation of SAWs that affect blood flow within vessels or other optoacoustic techniques. It may be that the same pulsed laser apparatus such as that shown in FIG. 8 is used for both optoacoustic flow control and the SAW generation. This reduces the complexity of the device.

Although the order in which the vascular sections are investigated is shown systematically in the priority column 640, the disclosure is not so limited. Instead, regions of interest may be defined either arithmetically or through a user interface to more rapidly determine the properties of vessels in areas relevant to the current task of the endoscope system. This may be achieved by identifying a point of interest such as a cut or a bleed and then vessels within that region of interest will be segmented and analysed as above. This will allow the depth of the cut causing the bleed to be analysed.

In the above example, where optoacoustic forces have been applied as the flow modifying pulse 415, this may generate undesirable SAWs or other artefacts which interfere with the vessel depth measurement. In this case, interference avoidance measures such as minimum distance from the area under investigation where the flow modifying pulse has been applied can be implemented. Further, it is possible to phase the SAWs so as to avoid interaction with the undesirable artefacts.

Although the above discusses applying multiple SAWs to the area under investigation, it may be desirable to apply a single SAW with multiple image capture events to avoid the exposure of the tissue to unnecessary SAW waves.

Various embodiments of the present disclosure are defined by the following numbered clauses:

1. A medical imaging system including circuitry configured to: apply a surface acoustic wave to tissue to interact with vessel; capture an image of the tissue when the surface acoustic wave interacts with the vessel; and identify a property of the vessel from the captured image.

2. A method according to clause 1, wherein the circuitry is configured to apply the surface acoustic wave when the vessel is dilated with a pulse of liquid.

3. A system according to clause 1 or 2, wherein the circuitry is configured to apply a flow modifying pulse to the tissue, the flow modifying pulse being configured to modify the pulse of liquid to increase the dilation of the vessel.

4. A system according to clause 3, wherein the circuitry is configured to apply the flow modifying pulse in a direction opposing the flow of the liquid in the vessel.

5. A system according to clause 3, wherein the circuitry is configured to apply a further flow modifying pulse to the tissue, the further flow modifying pulse being configured to further modify the pulse of liquid to further increase the dilation of the vessel.

6. A system according to clause 3, wherein the circuitry is configured to apply the flow modifying pulse for a period of time, the period of time being selected to reduce the flow of the liquid through the vessel.

7. A system according to any preceding clause, wherein the circuitry is further configured to generate a single surface acoustic wave to interact with the vessel; capture a plurality of images of the surface acoustic wave and identify a property of the vessel from a comparison of the plurality of captured images.

8. A system according to any preceding clause, whereby the property is the depth of the vessel within the tissue.

9. A system according to any preceding clause, wherein the circuitry is configured to apply a laser speckle pattern to the tissue as the surface acoustic wave is applied; and identify the property of the vessel from the captured speckle pattern.

10. A system according to any preceding clause, wherein the circuitry is provided in an endoscope.

11. A system according to any preceding clause wherein the vessel is vasculature and the liquid is blood.

12. A system according to any preceding clause, wherein the circuitry includes wave application circuitry configured to apply the surface acoustic wave to the tissue and imaging circuitry configured to capture an image of the tissue.

13. A medical imaging method including applying a surface acoustic wave to the tissue to interact with a vessel; capturing an image of the tissue when the surface acoustic wave interacts with the vessel; and identifying a property of the vessel from the captured image.

14. A method according to clause 13, including applying the surface acoustic wave when the vessel is dilated with a pulse of liquid.

15. A method according to clause 13 or 14, including applying a flow modifying pulse to the tissue, the flow modifying pulse being configured to modify the pulse of liquid to increase the dilation of the vessel.

16. A method according to clause 15, including applying the flow modifying pulse in a direction opposing the flow of the liquid in the vessel.

17. A method according to clause 15, including applying a further flow modifying pulse to the tissue, the further flow modifying pulse being configured to further modify the pulse of liquid to further increase the dilation of the vessel.

18. A method according to clause 15, including applying the flow modifying pulse for a period of time, the period of time being selected to reduce the flow of the liquid through the vessel.

19. A method according to any one of clause 13 to 18, including generating a single surface acoustic wave to interact with the vessel; capturing a plurality of images of the surface acoustic wave and identifying a property of the vessel from a comparison of the plurality of captured images.

20. A method according to any one of clause 13 to 19, whereby the property is the depth of the vessel within the tissue.

21. A method according to any one of clause 13 to 20, including applying a laser speckle pattern to the tissue as the surface acoustic wave is applied; and identifying the property of the vessel from the captured speckle pattern.

22. A method according to any one of clause 13 to 21 wherein the vessel is vasculature and the liquid is blood.

23. A computer program product including computer readable instructions which, when loaded onto a computer, configures the computer to perform a method according to any one of clause 13 to 22.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

Surface Acoustic Waves (SAWs)

A SAW is a wave that travels along the interface between two different elastic materials such as soft tissue and air. They have applications in many areas, being used for both sensing and actuation, but of particular relevance to the proposed invention is their application to depth sensing and mechanical property investigation.

Different frequency components of broadband SAWs propagate at different depths (approximately 1 wavelength from the surface) within a medium and propagate at different speeds based on the stiffness of the material. The phase velocity (speed of propagation of a certain frequency wave component) of the long wavelength SAW components will therefore be determined primarily by the deeper layers, whereas the shorter wavelength phase velocities will be determined by the properties of the surface layers. This can be used to evaluate the depth and elasticity of material layers with distinct mechanical properties. This technique allows the characterisation of different layers down to a reported 3.4 mm. These evaluation systems also have a minimum depth at which measurements can be made which is defined by the highest frequency components of a SAW that can be measured by the system.

SAWs can also be used to produce forces in fluids, or for their ability to alter the mechanical properties of fluids such as blood. Consequently, they are commonly employed in microfluidic actuation systems.

SAWs can be generated by creating an impulsive force on a surface, which can be achieved by several possible means including piezoelectric transducers in contact with a surface, focused ultrasound and pulsed laser generation (photoacoustic) techniques. With any of these techniques, a range of parameters may be used to create SAWs with tuneable properties.

Photoacoustic Techniques

Photoacoustic techniques use a short, high amplitude bursts of laser light which are highly absorbed by the target substrate to generate a rapid thermal expansion. If absorption occurs on a material surface, this rapid expansion creates longitudinal and transverse waves that travel through the body of the target material, as well as SAWs that propagate along the surface in all directions. Multiple sources or shaped single sources of SAWs can be used to generate shaped wavefronts and focal points. Although the laser pulse can damage tissue if a high intensity is used (for example, generating SAWs that propagate longer distances), there are well characterised parametric guidelines to avoid this as well as new techniques that may prevent the damage.

By using a laser that is strongly absorbed by blood plasma components such as water, strong photoacoustic forces local to the beam absorption site have been shown to affect and control the flow of fluid and particles in small vessels, which is currently under investigation for in vivo flow cytometric applications.

Laser Speckle Contrast Imaging (LSCI)

LSCI is an inexpensive, full-field imaging technique that uses the interference pattern generated by coherent light as it reflects and scatters off objects at different depths in a material, causing constructive and destructive interference. Any movement within the image will change the speckle pattern, making it a sensitive tool for imaging blood flow even down to the microvasculature. Movement of the target or imager/laser source can also change the speckle pattern which is a source of noise, though this can be largely corrected for, even using freely moving sources like endoscopes.

The ability to image the entire focal field at a single instance removes the requirement for laser scanning or high-speed photography, allowing LSCI to be performed with very low-cost equipment.

Further than just creating a sensitive 2D map of vasculature, several properties of the blood flow and vessels can be determined including flow direction and velocity, and estimations of static and dynamic vessel diameter. However, these estimations and the general application of LSCIs suffer from a lack of depth resolution.

As a SAW travels along a surface, it causes a small displacement of the tissue (<1 μm). This movement can be detected by LSCI techniques, and through further analysis of the speckle pattern, wave properties such as wave speed, wavelength and attenuation length can be measured.

1. A medical imaging system including circuitry configured to: apply a surface acoustic wave to tissue to interact with vessel; capture an image of the tissue when the surface acoustic wave interacts with the vessel;
and identify a property of the vessel from the captured image.

2. A method according to claim 1, wherein the circuitry is configured to apply the surface acoustic wave when the vessel is dilated with a pulse of liquid.

3. A system according to claim 1, wherein the circuitry is configured to apply a flow modifying pulse to the tissue, the flow modifying pulse being configured to modify the pulse of liquid to increase the dilation of the vessel.

4. A system according to claim 3, wherein the circuitry is configured to apply the flow modifying pulse in a direction opposing the flow of the liquid in the vessel.

5. A system according to claim 3, wherein the circuitry is configured to apply a further flow modifying pulse to the tissue, the further flow modifying pulse being configured to further modify the pulse of liquid to further increase the dilation of the vessel.

6. A system according to claim 3, wherein the circuitry is configured to apply the flow modifying pulse for a period of time, the period of time being selected to reduce the flow of the liquid through the vessel.

7. A system according to claim 1, wherein the circuitry is further configured to generate a single surface acoustic wave to interact with the vessel; capture a plurality of images of the surface acoustic wave and identify a property of the vessel from a comparison of the plurality of captured images.

8. A system according to claim 1, whereby the property is the depth of the vessel within the tissue.

9. A system according to claim 1, wherein the circuitry is configured to apply a laser speckle pattern to the tissue as the surface acoustic wave is applied; and identify the property of the vessel from the captured speckle pattern.

10. A system according to claim 1, wherein the circuitry is provided in an endoscope.

11. A system according to claim 1 wherein the vessel is vasculature and the liquid is blood.

12. A system according to claim 1, wherein the circuitry comprises wave application circuitry configured to apply the surface acoustic wave to the tissue and imaging circuitry configured to capture an image of the tissue.

13. A medical imaging method including applying a surface acoustic wave to the tissue to interact with a vessel; capturing an image of the tissue when the surface acoustic wave interacts with the vessel; and
identifying a property of the vessel from the captured image.

14. A method according to claim 13, including applying the surface acoustic wave when the vessel is dilated with a pulse of liquid.

15. A method according to claim 13, including applying a flow modifying pulse to the tissue, the flow modifying pulse being configured to modify the pulse of liquid to increase the dilation of the vessel.

16. A method according to claim 15, including applying the flow modifying pulse in a direction opposing the flow of the liquid in the vessel.

17. A method according to claim 15, including applying a further flow modifying pulse to the tissue, the further flow modifying pulse being configured to further modify the pulse of liquid to further increase the dilation of the vessel.

18. A method according to claim 15, including applying the flow modifying pulse for a period of time, the period of time being selected to reduce the flow of the liquid through the vessel.

19. A method according to claim 13, including generating a single surface acoustic wave to interact with the vessel; capturing a plurality of images of the surface acoustic wave and identifying a property of the vessel from a comparison of the plurality of captured images.

20. A method according to claim 13, whereby the property is the depth of the vessel within the tissue.

21. A method according to claim 13, including applying a laser speckle pattern to the tissue as the surface acoustic wave is applied; and
identifying the property of the vessel from the captured speckle pattern.

22. A method according to claim 13 wherein the vessel is vasculature and the liquid is blood.

23. A computer program product including computer readable instructions which, when loaded onto a computer, configures the computer to perform a method according to claim 13.
